(19) United States
(12) Patent Application Publication
(10) Pub. No.: US 2008/0058688 A1
(43) Pub. Date: Mar. 6, 2008

(54) Air-cushioned arm pad

(76) Inventor: Pang-Ching Chiang, Taipei (TW)

Correspondence Address: RABIN & Berdo, PC, 1101 14TH STREET, NW, SUITE 500, WASHINGTON, DC, 20005, US

(21) Appl. No.: 11/512,135

(22) Filed: Aug. 30, 2006

(51) Int. Cl. A61F 5/00

(52) U.S. Cl. 602/20

(57) ABSTRACT

An arm pad includes a first layer, a second layer securely attached to the first layer and a flat spring securely sandwiched between the first layer and the second layer. There is provided with a third layer sandwiched between the first layer and the second layer. The flat spring is securely mounted on top of the third layer so as to provide additional force to the arm when the arm is stretching or retracting.

### BACKGROUND OF THE INVENTION

1. Field of the Invention

The present invention relates to an arm pad, and more particularly to an arm pad having therein a flat spring to provide additional force to the arm when the arm is extending or retracting in a ball game.

2. Description of the Prior Art

The existing arm pad is provided with an elongated strap and Velcro® on opposite end sides of the strap such that the user is able to apply the arm pad on a portion of the arm to prevent injury to the arm muscles. Particularly, the ball players require a lot of arm stretching and retracting movements, which often is the cause for the ball players to suffer from arm injuries.

With reference to FIG. 7, the conventional arm pad (6) is provided with an elongated strap (61) and a ring (62) securely mounted on the elongated strap (61) so that the user is able to use the ring (62) to adjust the tightness of the arm pad (6) on the arm. Because the conventional arm pad (6) can only provide tightness to the arm, users of the conventional arm pad often do not wear the arm pad, unless it is absolutely necessary. Although the conventional arm pad does have the ability to provide support to the arm muscles and prevent the arm muscles from injuries, the conventional arm pad is not comfortable when wearing on the arm.

To overcome the shortcomings, the present invention tends to provide an improved arm pad to mitigate the aforementioned problems.

### SUMMARY OF THE INVENTION

The primary objective of the present invention is to provide an improved arm pad having therein air pouches so that not only tightness effect is provided to the arm, but also comfort effect is provided to the user.

Another objective of the present invention is that an elongated resilient element such as a spring is provided to the arm pad to provide additional assistance to the user especially when the user is stretching or retracting the arm.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of the arm pad of the present invention;

FIG. 2 is an exploded perspective view of a second embodiment of the arm pad of the present invention;

FIG. 3 is a top plan view showing the combined arm pad of the present invention;

FIG. 4 is a partially cross sectional view of the arm pad in FIG. 3 taken from line 4-4;

FIG. 4A is a partially cross sectional view of the arm pad of the second embodiment of the present invention;

FIG. 4B is an exploded perspective view of the arm pad of a third embodiment of the present invention;

FIG. 5 is a schematic perspective view showing the application of the arm pad with an elongated strap;

FIG. 6 is an exploded perspective view of another embodiment of the present invention; and

FIG. 7 is an operational view showing the application of the conventional arm pad.

### DETAILED DESCRIPTION OF THE PREFERRED EMBODIMENT

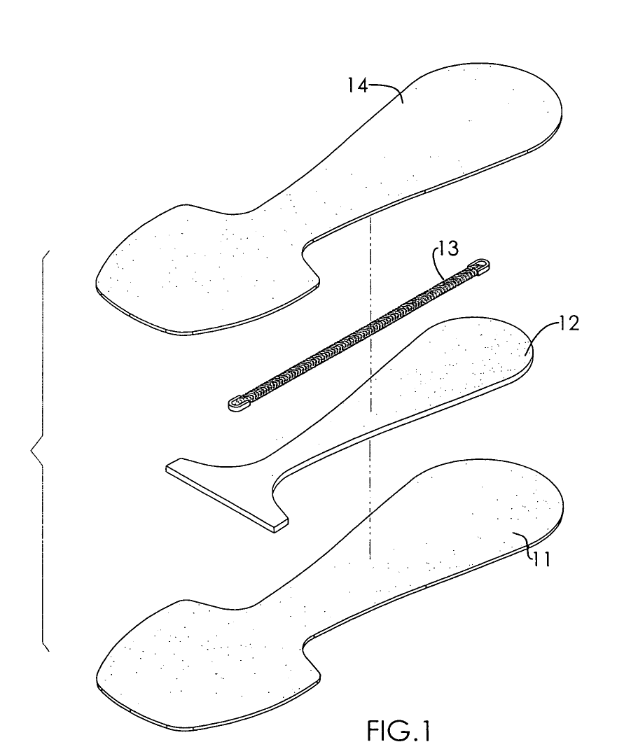

With reference to FIG. 1, it is noted that the arm pad in accordance with the present invention includes a first layer (11), a second layer (12), a flat spring (13) and a third layer (14). When the arm pad of the present invention is assembled, the second layer (12) is securely mounted on top of the first layer (11). The flat spring (13) is securely mounted on top of the second layer (12) and then the third layer (14) is securely mounted on top of the first layer (11) to sandwich the second layer (12) as well as the flat spring (13) therebetween.

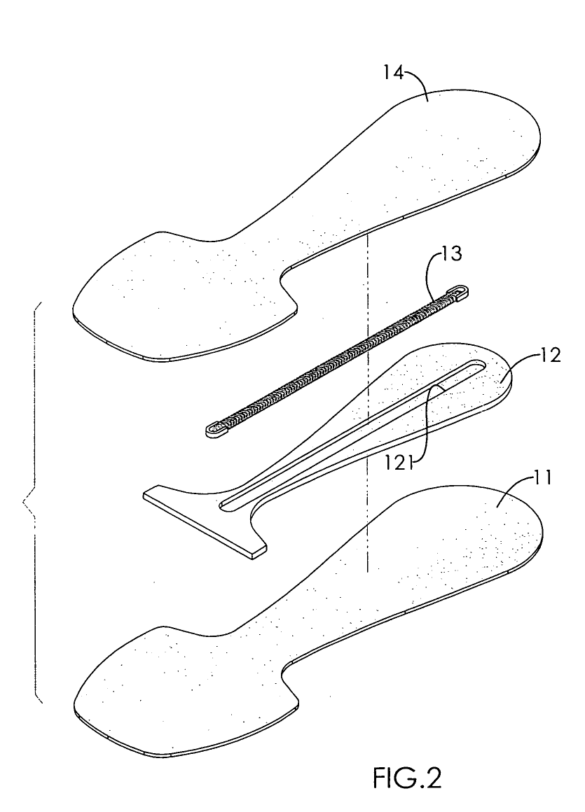

With reference to FIG. 2, it is noted that the second layer (12) is provided with an elongated recess (121) corresponding to the flat spring (13) so as to receive therein the flat spring (13).

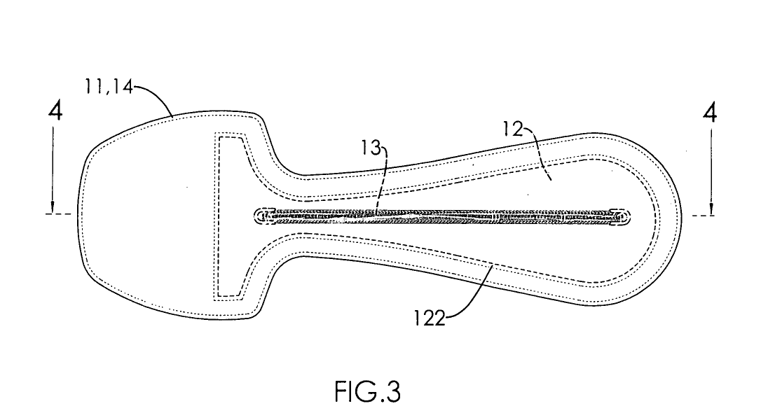

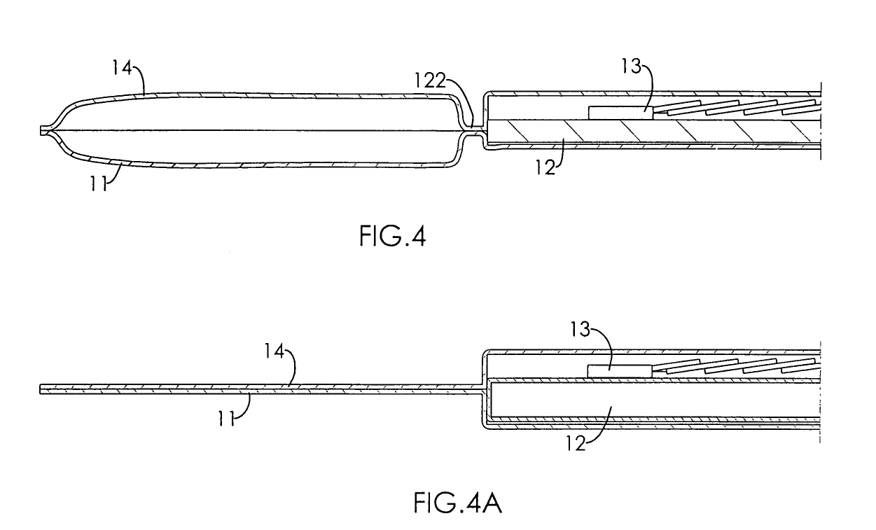

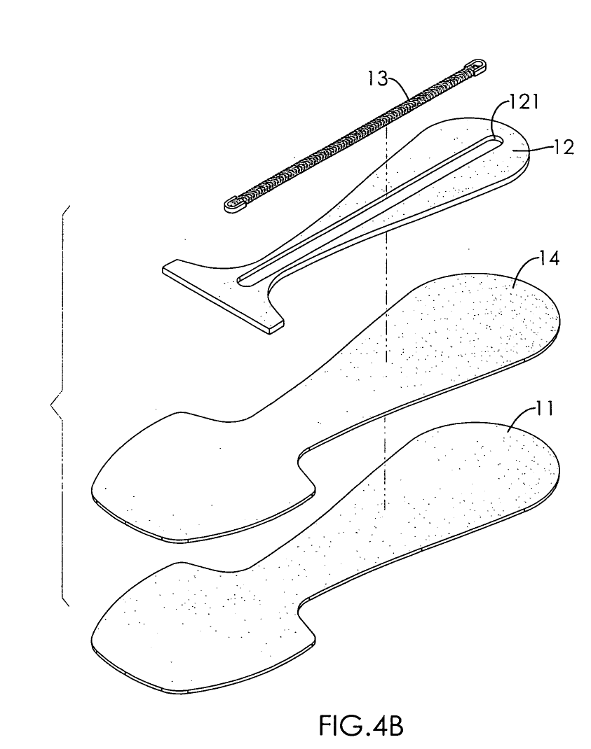

With reference to FIGS. 3 and 4, it is to be noted that in the first embodiment of the present invention, the combination of the first layer (11), the second layer (12) and the third layer (14) may be finished via gluing, sewing or the like so long as they are securely connected to one another. In this embodiment, the combination therebetween is based on sewing. Therefore, it is noted that the first layer (11) and the third layer (14) are full of gas and the second layer (12) is just a flat pad with the flat spring (130 securely sandwiched between the second layer (12) and the third layer (14). A seam (122) is formed along a contour of the second layer (12), where there is no gas in the seam (122).

With reference to FIG. 4A, it is noted that in this embodiment, the first layer (11) and the third layer (14) are flat layers and the second layer (12) is full of gas.

With reference to FIG. 4B, it is to be noted that the first layer (11) and the third layer (14) are securely connected together and then the second layer (12) is mounted on top of the third layer (14) with the flat spring (13) received in the elongated recess (121) in the second layer (12). It is noted that even though the second layer (12) is on top of the third layer (14), the second layer (12) may be provided with gas therein while the first layer (11) and the third layer (14) are not provided with gas.

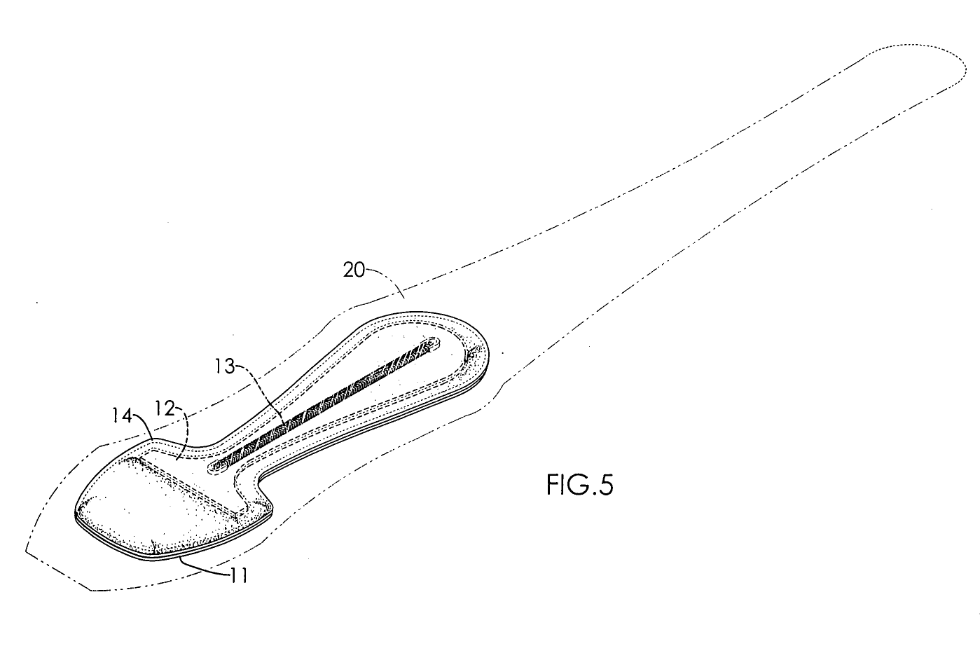

With reference to FIG. 5, when the arm pad of the present invention is in application, an elongated strap (20) is provided to the combination of the first layer (11), the second layer (12) and the third layer (14) so that the user is able to use the elongated strap (20) to mount the arm pad around the arm to provide support to the arm. In addition, the flat spring (13) provided on top of the second layer (12) is able to provide additional force to the arm especially when the arm is stretching or retracting.

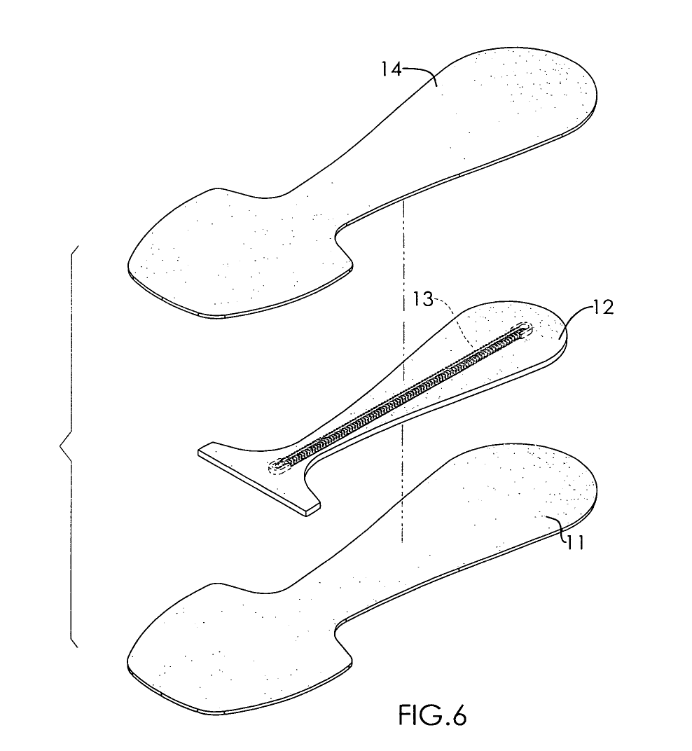

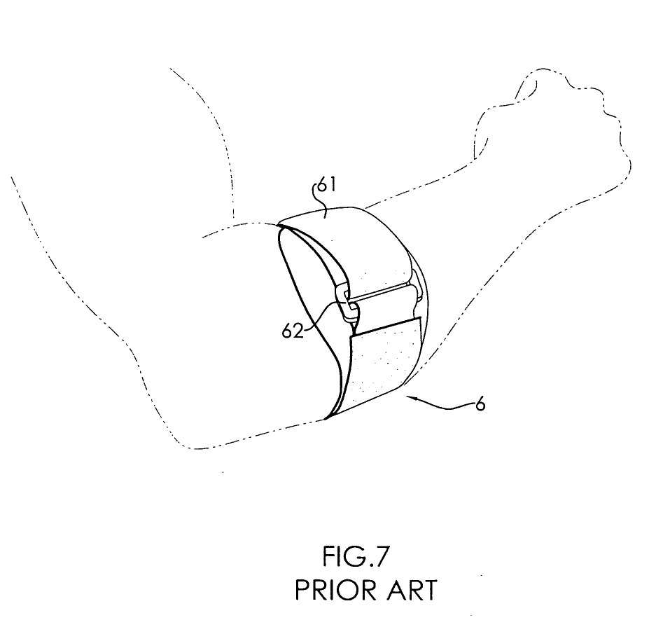

With reference to FIG. 6, it is to be noted that the flat spring (13) may be injection molded inside the second layer (12). Also, the flat spring (13) may be replaced with other resilient elements.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An arm pad comprising:
a first layer;
a second layer securely attached on top of the first layer;
a flat spring securely mounted on top of the second layer; and
a third layer mounted on top of the second layer to sandwich the flat spring with the second layer.

2. The arm pad as claimed in claim 1, wherein the first layer and the third layer are full of gas.

3. The arm pad as claimed in claim 2, wherein the second layer has an elongated recess corresponding to and receiving the flat spring therein.

4. The arm pad as claimed in claim 1, wherein the second layer is full of gas and has an elongated recess corresponding to and receiving the flat spring therein.

5. An arm pad comprising:
a first layer full of gas;
a second layer mounted on top of the first layer;
a flat spring securely mounted on top of the second layer; and
a third layer full of gas and mounted on top of the second layer to sandwich the flat spring with the second layer.

6. The arm pad as claimed in 5, wherein the second layer has an elongated recess corresponding to and receiving the flat spring therein.

7. An arm pad comprising:
a first layer;
a second layer full of gas and securely attached to the first layer;
a flat spring securely mounted on top of the second layer; and
a third layer securely mounted on top of the second layer to sandwich the flat spring with the second layer.

8. The arm pad as claimed in claim 7, wherein the second layer has an elongated recess corresponding to and receiving the flap spring therein.

9. An arm pad comprising:
a first layer;
a flap spring mounted on top of the first layer; and
a second layer mounted on top of the first layer to sandwich the flap spring with the first layer.

10. The arm pad as claimed in claim 9, wherein the first layer and the second layer are full of gas.

11. The arm pad as claimed in claim 10, wherein a third layer is sandwiched between the first layer and the second layer, the flap spring is received in an elongated recess in the third layer.

12. The arm pad as claimed in claim 9, wherein the third layer is full of gas and has an elongated recess corresponding to and receiving therein the flap spring.

13. An arm pad comprising:
a first layer;
a second layer securely mounted on top of the first layer; and
a flap spring securely mounted on top of the second layer.

14. The arm pad as claimed in claim 13, wherein the first layer and the second layer are full of gas.

15. The arm pad as claimed in claim 14, wherein a third layer mounted on top of the second layer and has an elongated recess defined to correspond to and receive therein the flat spring.

16. The arm pad as claimed in claim 15, wherein the third layer is full of gas and has an elongated recess defined to correspond to and receive therein the flat spring.

17. An arm pad comprising:
a first layer;
a second layer full of gas and securely mounted on top of the first layer;
a flap spring mounted inside the second layer; and
a third layer securely mounted on top of the second layer to sandwich the flap spring with the second layer.

18. The arm pad as claimed in claim 17, wherein the second layer has an elongated recess to receive therein the flap spring.

19. The arm pad as claimed in claim 17, wherein the flap spring may be an elastic object or any other object that is resilient.
